(19) United States
(12) Patent Application Publication
(10) Pub. No.: US 2025/0003867 A1
(43) Pub. Date: Jan. 2, 2025

(54) CHEMICAL SENSORS USING COPPER HIGH-ASPECT STRUCTURES (CuHARS) AND METHODS FOR USING SAME

(71) Applicant: LOUISIANA TECH RESEARCH CORPORATION, Ruston, LA (US)

(72) Inventor: Mark A. DECOSTER, Ruston, LA (US)

(73) Assignee: LOUISIANA TECH RESEARCH CORPORATION, Ruston, LA (US)

(21) Appl. No.: 18/755,085

(22) Filed: Jun. 26, 2024

Related U.S. Application Data
(60) Provisional application No. 63/523,806, filed on Jun. 28, 2023.

(51) Int. Cl. G01N 21/21; G01N 33/208

(52) U.S. Cl. CPC G01N 21/211; G01N 33/208

(57) ABSTRACT

Disclosed herein are chemical sensors that include high aspect copper structures and methods for using the chemical sensor to detect the presence of chemical species. In a specific embodiment, the chemical sensor can include: a substrate; a film, where the film includes one or more copper cystine biocomposites, and where the film is at least partially disposed on one or more surfaces of the substrate; a light source, where the light source irradiates the one or more copper structures; and a detector, where the detector receives light reflected from the one or more copper cystine biocomposites, and where the detector detects a change in light polarization when the one or more copper structures contact one or more chemical species.

### CROSS-REFERENCE TO RELATED APPLICATIONS

This application claims the benefit of U.S. Provisional Patent Application No. 63/523,806, filed Jun. 28, 2023, which is incorporated by reference herein in its entirety.

### BACKGROUND

### Field

Disclosed herein are chemical sensors that include copper high aspect structures and methods for detecting chemical species using changes in light polarization.

### Description of the Related Art

As industrial and commercial activities continue to accelerate, many manmade chemical species have found their way into the environment, heightening concern about human health and safety. Sensor methods and sensor films for quantification of volatile and nonvolatile chemical species in fluids are known in the art. However, these sensors often involve multi-step linked reactions and expensive equipment for detection. For example, many sensors require fluorescence or colorimetric labels and expensive illumination light sources and filters.

Consequently, there is a need for new chemical sensors that do not require labeling technology nor expensive detection equipment.

### SUMMARY

Provided herein are high aspect ratio copper structures and methods for using them as chemical sensors. In a specific embodiment, the chemical sensor can include: a substrate; a film, where the film includes one or more copper cystine biocomposites, and where the film is at least partially disposed on one or more surfaces of the substrate; a light source, where the light source irradiates the one or more copper structures; and a detector, where the detector receives light reflected from the one or more copper cystine biocomposites, and where the detector detects a change in light polarization when the one or more copper structures contact one or more chemical species.

In another specific embodiment, a method for sensing the presence of a chemical species can include: making one or more copper structures, where the method of making the one or more copper structures includes: contacting one or more copper species, one or more cystines, one or more carrier fluids to make a first mixture; agitating the first mixture to make an agitated first mixture; and incubating the agitated first mixture to make the one or more copper structures; applying a film of the one or more copper structures to one or more surfaces of a substrate; irradiating the one or more copper structures with a light source; detecting a change in light polarization when the one or more copper structures contact the chemical species.

In another specific embodiment, a method for a chemical sensor, where the method includes: making one or more copper cystine biocomposites, where a method of making the one or more copper cystine biocomposites includes: contacting one or more copper species, one or more cystines, one or more carrier fluids to make a one or more copper cystine biocomposites; applying a coating of the one or more copper cystine biocomposites to one or more surfaces of a substrate; contacting the one or more copper cystine biocomposites with a known chemical species; irradiating the one or more copper structures that is in contact with the known chemical species with a light source; measuring a first polarization of a reflected light from the one or more copper cystine biocomposites that is in contact with a known chemical species; contacting the one or more copper cystine biocomposites with an unknown chemical species; irradiating the one or more copper structures that is contact with the unknown chemical species with a light source; measuring a second polarization of the reflected light from the one or more copper cystine biocomposites that is in contact with the unknown chemical species; comparing the first polarization with the second polarization.

### BRIEF DESCRIPTION OF THE DRAWINGS

The present disclosure can be better understood by referring to the following drawings. The drawings constitute a part of this specification and include exemplary embodiments of the high aspect ratio copper structures and methods, which may be embodied in various forms.

FIG. 1 shows an embodiment of a method for detecting chemical species using high aspect ratio copper structures.

FIG. 2 shows an embodiment of a method for making copper structures.

FIG. 3 shows a magnified image of an edge of a droplet of a copper composition that contains copper structures with (D+L)-cystine.

FIG. 4 shows a magnified image of an edge of a droplet of a copper composition that contains copper structures with D-cystine.

FIG. 5 shows a magnified image of an edge of a droplet of a copper composition that contains copper structures with L-cystine.

FIG. 6 shows an image of dried droplets of three formulations of CuHARS made from D-cystine, L-cystine, and racemic (D+L)-cystine on a microscope slide and illuminated through a polarization film.

### DETAILED DESCRIPTION

The chemical sensor can include a fast chemical sensor that uses polarized light to sense the intrinsic changes in the properties of copper high-aspect ratio structures (CuHARS) when they contact one or more chemical species. In one or more embodiments, the chemical sensor can include, but is not limited to: one or more copper structures, one or more copper cystine biocomposites, one or more copper compositions, one or more substrates, one or more films, one or more detectors, one or more lens, one or more mirrors, one or more cameras, one or more central processing units, one or more memories, one or more electronic circuitry, one or more monitors, one or more receivers, one or more transmitters, one or more light sources, and one or more ocular members. The chemical sensors can provide high sensitivity and broad dynamic range. In an embodiment, the chemical sensor using CuHARS can solve the problem of slow and expensive sensing by using economical and quantifiable polarization films combined with available white light microscopy and imaging. In another embodiment, the copper structures can have intrinsic polarization properties, allowing for label-free detection. Further, it has been demonstrated that detection is possible without the need of built in microscopy polarization filters, and can be carried out using white-light microscopy illumination sources combined with polarization films.

In an embodiment, the chemical sensor can be an optical sensor. For example, the optical sensor can detect changes in light polarization from light reflected from the one or more copper structures when the one or more copper structures contact a chemical species. In an embodiment, the chemical sensor can be used in white light microscopy, bright-field microscopy, scanning white light interference microscopy, phase contrast light microscopy, dark field light microscope polarized microscopy, confocal microscopy, phase contrast microscopy, differential interference contrast microscopy. In another embodiment, the chemical sensor can detect one or more chemical species in a gas, liquid, solid, and mixtures thereof.

The one or more copper compositions and/or copper structures can be used for a variety of commercial applications. For example, the uses for the copper compositions and/or copper structures can include, but are not limited to: fast sensing of chemical interactions through liquid or vapor presence, educational kits and educational supplies, research tools for basic science research, research tools for engineering research and quality control devices, diagnostic kits, environmental sensors, safety sensors, chemical sensors, chemosensors, and combinations thereof.

The one or more copper structures can include, but is not limited to: one or more high aspect ratio copper structures, one or more copper biocomposites, one or more copper cysteine biocomposites, one or more copper cystine biocomposites, one or more copper nanoparticles, one or more copper sulfates, and mixtures thereof. The one or more cystines can include, but is not limited to: one or more L-cystines, one or more D-cystines, one or more R-cystines, one or more S-cystines, and mixtures thereof. The one or more cystines (dimer form) can include, but is not limited to: one or more L-cystines, one or more R-cysteines, one or more S-cysteines, one or more D-cysteines, one or more L-cysteines, and mixtures thereof. Compared to nanoparticles, higher-aspect ratio of the one or more copper structures can provide biophysical benefits such as reversal of the coffee-ring effect and may also serve as connectors for cellular scaffolding or device construction.

The one or more copper structures can have a content of the one or more cystines and/or one or more cysteines that can vary widely. For example, the one or more copper structure can have the one or more cystines and/or one or more cysteines content from low of about 0.001 wt %, about 0.01 wt %, or about 0.1 wt %, to a high of about 90.0 wt %, about 95.0 wt %, or about 99.999 wt %. In another example, the one or more copper structure can have the one or more cystines and/or one or more cysteines content from about 0.001 wt % to about 99.999 wt %, about 0.001 wt % to about 0.01 wt %, about 0.001 wt % to about 0.1 wt %, about 0.01 wt % to about 0.1 wt %, about 0.1 wt % to about 1.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the one or more cystines and/or one or more cysteines in the copper structures can be based on the total weight of the copper composition, or based on the one or more copper structures, the one or more solvents and/or carrier fluids, and the one or more additives.

The copper structures and/or copper particles can include, but is not limited to: nano-sized copper structures, to micro-sized copper structures, and mixtures thereof. The copper structures and/or copper particles can have a diameter that varies widely. For example, the copper structures and/or copper particles can have a diameter from a low about 50 nm, about 60 nm, or about 80 nm, to a high of about 140 μm, about 150 μm, or about 200 μm. In another example, the copper structures and/or copper particles can have a diameter from about 60 nm to about 60 microns, 50 nm to about 200 μm, about 50 nm to about 100 nm, about 60 nm to about 500 nm, about 60 nm to about 10 μm, about 65 nm to about 20 μm, about 70 nm to about 110 nm, about 75 nm to about 120 nm, about 80 nm to about 150 nm, about 80 nm to about 150 μm, about 80 nm to about 200 μm, or about 100 nm to about 180 μm.

The copper structures and/or copper particles can have a length that varies widely. For example, the copper structures and/or copper particles can have a length from a low about 50 nm, about 60 nm, or about 80 nm, to a high of about 140 μm, about 150 μm, or about 500 μm. In another example, the copper structures and/or copper particles can have a length from about 50 nm to about 200 μm, about 50 nm to about 500 μm, about 50 nm to about 100 nm, about 60 nm to about 500 nm, about 60 nm to about 10 μm, about 65 nm to about 20 μm, about 70 nm to about 110 nm, about 75 nm to about 120 nm, about 80 nm to about 150 nm, about 80 nm to about 150 μm, about 80 nm to about 200 μm, about 100 nm to about 180 μm, about 90 μm to about 170 μm, about 100 μm to about 180 μm, about 120 μm to about 220 μm, about 150 μm to about 250 μm, or about 200 μm to about 500 μm. In another example, the copper structures and/or copper particles can have a length of greater than about 150 μm, greater than about 200 μm, or greater than about 250 μm.

The copper structures and/or copper particles can have an average aspect ratio that varies widely. For example, the copper structures and/or copper particles can average aspect ratio from a low of about 1, about 10, about 30, to a high of about 110, about 130, or about 180. For example, the copper structures and/or copper particles average aspect ratio from about 1 to about 10, 80 to about 180, about 90 to about 120, about 90 to about 100, about 90 to about 100, about 100 to about 110, about 95 to about 115, or about 95 to about 155.

The one or more copper compositions can include the one or more copper structures, one or more solvents and/or carrier fluids, and one or more additives. For example, the copper compositions can have a content of the one or more copper structures from a low of about 0.001 wt %, about 0.01 wt %, or about 0.1 wt %, to a high of about 90.0 wt %, about 95.0 wt %, or about 99.999 wt %. In another example, the copper compositions can have a content of the one or more copper structures from about 0.001 wt % to about 99.999 wt %, about 0.001 wt % to about 0.01 wt %, about 0.001 wt % to about 0.1 wt %, about 0.01 wt % to about 0.1 wt %, about 0.1 wt % to about 1.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the copper structures in the copper composition can be based on the total weight of the copper composition; or based on the one or more copper structures, the one or more solvents and/or carrier fluids, and the one or more additives.

The one or more solvents and/or carrier fluids can include, but is not limited to: water, hexanes, toluene, methanol, ethanol, propanol, isopropanol, acetone, acetonitrile, chloroform, diethyl ether, dimethyl sulfoxide, methylene chloride, dimethyl formamide, ethylene glycol, propylene glycol, triethylamine, tetrahydrofuran, and mixtures thereof.

The copper compositions can have a content of the one or more solvents and/or carrier fluids from a low of about 0.001 wt %, about 0.01 wt %, or about 0.1 wt %, to a high of about 90.0 wt %, about 95.0 wt %, or about 99.999 wt %. In another example, the copper compositions can have a content of the one or more solvents and/or carrier fluids from about 0.001 wt % to about 99.999 wt %, about 0.001 wt % to about 0.01 wt %, about 0.001 wt % to about 0.1 wt %, about 0.01 wt % to about 0.1 wt %, about 0.1 wt % to about 1.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the one or more solvents and/or carrier fluids in the copper composition can be based on the total weight of the copper composition, or based on the one or more copper structures, the one or more solvents and/or carrier fluids, and the one or more additives.

For example, the copper compositions can have a content of the one or more additives from a low of about 0.001 wt %, about 0.01 wt %, or about 0.1 wt %, to a high of about 90.0 wt %, about 95.0 wt %, or about 99.999 wt %. In another example, the copper compositions can have a content of the one or more additives from about 0.001 wt % to about 99.999 wt %, about 0.001 wt % to about 0.01 wt %, about 0.001 wt % to about 0.1 wt %, about 0.01 wt % to about 0.1 wt %, about 0.1 wt % to about 1.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the one or more solvents and/or carrier fluids in the copper composition can be based on the total weight of the copper composition, or based on the one or more copper structures, the one or more solvents and/or carrier fluids, and the one or more additives.

The one or more copper composition can have a solids content that varies widely. For example, the one or more copper composition can have solids content from a low of about 0.1 wt %, about 10.0 wt %, or about 20.0 wt %, to a high of about 90.0 wt %, about 95.0 wt %, or about 99.9 wt %. In other example, the copper composition can have a solids content from about 0.1 wt % to about 99.9 wt %, about 1.0 wt % to about 99.0 wt %, about 10.0 wt % to about 90.0 wt %, about 10.0 wt % to about 20.0 wt %, about 20.0 wt % to about 30.0 wt %, about 25.0 wt % to about 75.0 wt %, about 20.0 wt % to about 80.0 wt %, about 20.0 wt % to about 30.0 wt %, about 20.0 wt % to about 60.0 wt %, about 30.0 wt % to about 40.0 wt %, about 30.0 wt % to about 70.0 wt %, about 40.0 wt % to about 60.0 wt %, about 45.0 wt % to about 55.0 wt %, about 40.0 wt % to about 50.0 wt %, about 69.0 wt % to about 75.0 wt %, about 68.0 wt % to about 82.0 wt %, about 72.0 wt % to about 86.0 wt %, about 50.0 wt % to about 73.0 wt %, about 33.0 wt % to about 48.0 wt %, about 60.0 wt % to about 70.0 wt %, about 71.0 wt % to about 81.0 wt %, about 20.0 wt % to 30.0 wt %, about 50.0 wt % to about 60.0 wt %, or about 70.0 wt % to about 80.0 wt %. The weight percent of the solids content in the copper composition can be based on the total weight of the copper composition, or based on the one or more copper structures, the one or more solvents and/or carrier fluids, and the one or more additives.

The one or more copper composition can have a viscosity that varies widely. For example, the one or more copper compositions can have a viscosity from a low of about 100 cP, about 1,000 cP, or about 100,000 cP, to a high of about 250,000 cP, about 900,000 cP, or about 2,500,000 cP. In another example, the one or more copper compositions can have a viscosity from about 100 cP to about 2,500,000 cP, about 1,000 cP to about 250,000 cP, about 2,500 cP to about 250,000 cP, about 2,500 cP to about 200,000 cP, about 10,000 cP to about 100,000 cP, about 10,000 cP to about 50,000 cP, about 100,000 cP to about 250,000 cP, about 620,000 cP to about 850,000 cP, about 700,000 cP to about 750,000 cP, about 700,000 cP to about 800,000 cP, about 650,000 cP to about 855,000 cP, about 700,000 cP to about 800,000 cP, about 500,000 cP to about 1,000,000 cP, or about 500,000 cP to about 2,500,000 cP. The viscosity of the one or more copper compositions can be measured on a Brookfield viscosimeter, although other instruments can be used. The viscosity of the one or more copper compositions can be measured at various temperatures, such as 25° C., 40° C., 60° C., and 100° C.

The pH of the one or more copper composition can vary widely. For example, the one or more copper composition can have a pH from a low of about 0.1, about 1.0, about 2.0, to a high of about 12.0, about 13.0 or about 14.0. In another example, the one or more copper composition can have a pH from about 0.1 to about 13.9, about 4.0 to about 12.0, about 5.0 to about 10.0, about 7.5 to about 11.0, about 7.0 to about 10.0, about 8.0 to about 9.0, about 9.0 to about 10.0, about 8.0 to about 10.0, about 9.0 to about 11.0, or about 6.0 to about 9.0.

The one or more copper compositions and/or copper structures can show chemical changes, yielding changes of reflected light polarization, using small volumes of solutions of the one or more chemical species. For example, the one or more chemical species can be contacted with the one or more copper compositions and/or copper structures in a volume of solution from a low of about 2 μL, about 3 μL, or about 6 μL, to a high of about 1 mL, about 10 mL, or about 100 mL. In another example, the one or more chemical species can be contacted with the one or more copper compositions and/or copper structures in a volume of solution from about 2 μL to about 100 mL, about 2 μL to about 4 μL, about 3 μL to about 6 μL, about 4 μL to about 6 μL, about 4 μL to about 20 μL, about 100 μL to about 5 mL, about 1 mL to about 10 mL, or about 5 mL to about 100 mL.

The copper compositions and/or copper structures can include a zeta potential that varies widely. For example, the copper compositions and/or copper structures can include a zeta potential from a low of about-30 mV, about-20 mV, or about-10 mV, to a high of about 10 mV, about 20 mV, or about 30 mV. In another example, the copper compositions and/or copper structures can include a zeta potential from about-30 mV to about 30 mV, about-30 mV to about-20 mV, about-30 mV to about-10 mV, about-20 mV to about 20 mV, about-20 mV to about 0 mV, about-10 mV to about 0 mV, about-5 mV to about 5 mV, about 0 mV to about 10 mV, about 0 mV to about 20 mV, about 0 mV to about 30 mV, about 0 mV to about 5 mV, about 0 mV to about 10 mV, about 0 mV to about 20 mV, or about 0 mV to about 30 mV. In another example, the copper compositions and/or copper structures can have a net negative charge of about-24.3±2.2 mV, while the starting metal nanoparticles was measured at 43.3±2.4 mV.

The one or more substrates can include but is not limited to: glass, plastic, ceramic, polymer semiconductor, or combinations thereof. The one or more substrates can include, but is not limited to, a flat substrate. For example, the substrate can be flat enough to reflect incident light. A high sensitivity and a broader dynamic range can be attained if the substrate is highly reflective.

The one or more films can include, but is not limited to: the one or more copper compositions, the one or more copper structures, and mixtures thereof. In an embodiment, the films can include a light reflecting film and/or polarized film. The copper composition and/or copper structures can be applied to the one or more substrates in a thickness that can vary widely. For example, the copper composition and/or copper structures can be applied to the one or more substrates in a thickness from a low of about 50 nm, about 60 nm, or about 80 nm, to a high of about 1 mm, about 150 mm, or about 1 cm. In another example, the copper composition and/or copper structures can be applied to the one or more substrates in a thickness from about 50 nm to about 1 cm, about 50 nm to about 500 μm, about 50 nm to about 100 μm, about 100 μm to about 1 mm, about 1 mm to about 2 mm, about 1 mm to about 4 mm, or about 2 mm to about 1 cm. The one or more copper structures can provide highly-non aggregating material that can evenly coat surfaces for detection using polarized light.

The one or more light sources can include, but is not limited to halogen light, incandescent light, light emitting diode, and combination thereof. The one or more lens can include, but is not limited to: condenser lens and objective lens. In an embodiment, the condenser lens focuses light from the light source onto the sample potentially containing a chemical species, which is to be detected. In another embodiment, the objective lens can collect light from the sample and/or magnifies the image. In another embodiment, the light can be split, guided, collected, and/or collimated on the sensing element and/or detectors.

In an embodiment, the chemical the detection of the gaseous or liquid chemical species can include measuring changes in the light reflection characteristic from the one or more copper structures, such as changes in the polarization of the light.

In an embodiment, the one or more films of the one or more copper composition and/or copper structures can either react with or absorbs the one or more chemical species. In another embodiment, the chemical sensor can detect changes in light polarization to detect the one or more chemical species. The quantity of light reflected by the thin film of CuHARS on the substrate can be determined by many factors including: the incident angle of the light: the degree of polarization of the light; the thickness of the thin film, the refractive index of the thin film; the refractive indices of the media above and below the thin film, and other factors. The light reflected from the surface of the thin film interferes with the light reflected at the interface between the thin film and the substrate. This phenomenon of light interference can be dependent on the thickness and refractive index of the thin film.

In an embodiment, the one or more detectors and/or electronic circuitry can detect for the presence of a chemical species by making comparison between an electric signal that is indicative of the polarization and/or intensity of a reference light and an electric signal that is indicative of the polarization and/or intensity of the reflected light. In another embodiment, the one or more detectors and/or electronic circuitry can detect for the presence of a chemical species by making comparison between an electric signal that is indicative of the polarization and/or intensity of a standardized sample of a known chemical species and an electric signal that is indicative of the polarization and/or intensity of the reflected light from unknown chemical species.

In an embodiment, the one or more electronic circuitry can produce an output signal that responds linearly to the quantity of the chemical species to be detected. If the chemical sensor is configured to detect the presence of toxic, combustible, or flammable gases, vapors or solvents, the signal generated by the electronic circuitry can be used to trigger an alarm and/or perform on-off control over various switches or pipelines.

The one or more chemical species that can be detected by the chemical sensor can include, but is not limited to: microplastics, acids, bases, water vapor, hydrogen peroxide, nitric oxide, and mixtures thereof.

The quantity of light reflected by the thin film of CuHARS on the substrate can be determined by many factors including: the incident angle of the light: the degree of polarization of the light; the thickness of the thin film, the refractive index of the thin film; the refractive indices of the media above and below the thin film, and other factors. The light reflected from the surface of the thin film interferes with the light reflected at the interface between the thin film and the substrate. This phenomenon of light interference is largely dependent on the thickness and refractive index of the thin film.

In or more embodiments, the methods for using the copper composition and/or copper structures to detect a chemical species can include, but is not limited to: making one or more copper structures, where the method of making the one or more copper structures includes: contacting one or more copper species, one or more cystines, one or more carrier fluids to make a first mixture; agitating the first mixture to make an agitated first mixture; and incubating the agitated first mixture to make the one or more copper structures; applying a film of the one or more copper structures to one or more surfaces of a substrate; irradiating the one or more copper structures with a light source; detecting a change in light polarization when the one or more copper structures contact the chemical species. In an embodiment, a method for making the copper composition and/or copper structures can include, but is not limited to: a first mixture, second mixture, third mixture, fourth mixture, fifth mixture and more mixtures. In another embodiment, a method for using the copper composition and/or copper structures can include, but is not limited to: a first mixture, second mixture, third mixture, fourth mixture, fifth mixture and more mixtures.

In one or more embodiments, the methods for a chemical sensor can include making one or more copper cystine biocomposites, where a method of making the one or more copper cystine biocomposites includes: contacting one or more copper species, one or more cystines, one or more carrier fluids to make a one or more copper cystine biocomposites; applying a coating of the one or more copper cystine biocomposites to one or more surfaces of a substrate; contacting the one or more copper cystine biocomposites with a known chemical species; irradiating the one or more copper structures that is in contact with the known chemical species with a light source; measuring a first polarization of a reflected light from the one or more copper cystine biocomposites that is in contact with a known chemical species; contacting the one or more copper cystine biocomposites with an unknown chemical species; irradiating the one or more copper structures that is contact with the unknown chemical species with a light source; measuring a second polarization of the reflected light from the one or more copper cystine biocomposites that is in contact with the unknown chemical species; comparing the first polarization with the second polarization.

The copper structures can be made with different physical characteristics and polarization characteristics. These characteristics then change quickly depending upon the chemical addition to the material. In an embodiment, the method of making the copper composition and/or copper structures can be self-limiting. For example, when stoichiometry and the environmental conditions are chosen correctly, CuHARS self-assemble in a self-limiting manner. The reaction to form CuHARS may thus be terminated by simply removing the reaction vessel from heat, and moving it to a cooler temperature such as refrigeration (4° C.). In another embodiment, the copper structures can be made using green chemistry methodologies, which can be performed at body temperature or lower.

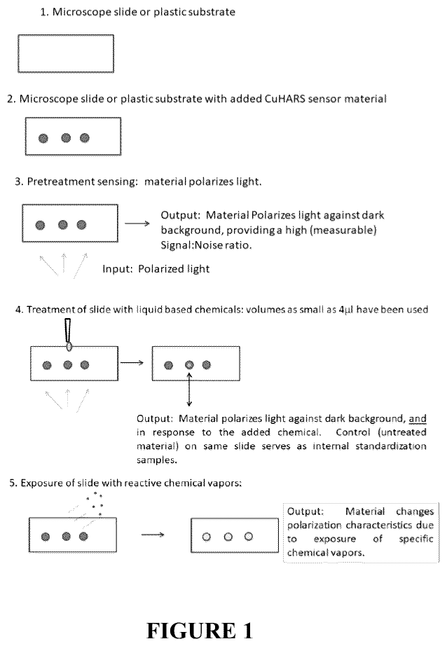

FIG. 1 shows an embodiment of a method for using the one or more copper compositions and/or copper structures to detect a chemical species. In step 1, a substrate is obtained. In step 2, a film of the one or more copper compositions and/or copper structures is applied to a surface of the substrate. In step 3, the one or more copper compositions and/or copper structures are illuminated with polarized light. The one or more copper compositions and/or copper structures polarizes light against a dark background, providing high signal to noise ratio. In step 4, the slide is treated with minimal volume of a chemical species, which can be used as a standard. Treated areas using the one or more copper compositions and/or copper structures (indicated with double-headed arrow) are compared to untreated areas on the same slide. In step 5, the one or more copper compositions and/or copper structures may also be exposed to volatile chemical species for detection. After exposure detection would be compared to untreated slides. Steps in applying invented materials with a microscope slide for detection of polarized light responsive patterns is shown.

The first mixture, second mixture, third mixture, fourth mixture, fifth mixture and more mixtures can have a viscosity that varies widely. For example, the first mixture, second mixture, third mixture, fourth mixture, and more mixtures can have a viscosity from a low of about 100 cP, about 1,000 cP, or about 100,000 cP, to a high of about 250,000 cP, about 900,000 cP, or about 2,500,000 cP. In another example, the first mixture, second mixture, third mixture, fourth mixture, fifth mixture, and more mixtures can have a viscosity from about 100 cP to about 2,500,000 cP, about 1,000 cP to about 250,000 cP, about 2,500 cP to about 250,000 cP, about 2,500 cP to about 200,000 cP, about 10,000 cP to about 100,000 cP, about 10,000 cP to about 50,000 cP, about 100,000 cP to about 250,000 cP, about 620,000 cP to about 850,000 cP, about 700,000 cP to about 750,000 cP, about 700,000 cP to about 800,000 cP, about 650,000 cP to about 855,000 cP, about 700,000 cP to about 800,000 cP, about 500,000 cP to about 1,000,000 cP, or about 500,000 cP to about 2,500,000 cP. The viscosity of the one or more mixtures can be measured on a Brookfield viscosimeter. The viscosity of the one or more mixtures can be measured at various temperatures, such as 25° C., 40° C., 60° C., and 100° C.

The pH of the first reaction mixture, second reaction mixture, third mixture, fourth mixture and more mixtures can have a pH vary widely. For example, the first reaction mixture, second reaction mixture, third mixture, fourth mixture and more mixtures can have a pH from a low of about 0.1, about 1.0, about 2.0, to a high of about 12.0, about 13.0 or about 14.0. In another example, the first reaction mixture, second reaction mixture, third mixture, fourth mixture and more mixtures can have a pH from about 0.1 to about 13.9, about 4.0 to about 12.0, about 5.0 to about 10.0, about 7.5 to about 11.0, about 7.0 to about 10.0, about 8.0 to about 9.0, about 9.0 to about 10.0, about 8.0 to about 10.0, about 9.0 to about 11.0, or about 6.0 to about 9.0.

The first mixture, first reaction mixture, second mixture, second reaction mixture, third mixture, third reaction mixture, fourth mixtures or more mixtures can be incubated and/or heated to a temperature from a low of about 0° C., about 15° C., and about 25° C., to a high of about 35° C., about 65° C., and about 200° C. For example, the first mixture, first reaction mixture, second mixture, second reaction mixture, third mixture, third reaction mixture, fourth mixtures or more mixtures can be heated to a temperature from about 25° C. to about 28° C., about 25° C. to about 35° C., about 25° C. to about 90° C., about 30° C. to about 45° C., about 35° C. to about 39° C., about 40° C. to about 90° C., about 43° C. to about 78° C., about 40° C. to about 90° C., about 100° C. to about 200° C. In another example, the first mixture, first reaction mixture, second mixture, second reaction mixture, third mixture, third reaction mixture, fourth mixtures or more mixtures can be at room temperature and/or ambient temperatures.

The first mixture, first reaction mixture, second mixture, second reaction mixture, third mixture, third reaction mixture, fourth mixtures or more mixtures can be agitated and/or incubated for a first reaction time, second reaction time, third reaction time, or more reaction times from a short time of about 15 s, about 120 s, or about 300 s, to a long time of about 1 h, about 24 h, or about 72 h. For example, first mixture, first reaction mixture, second mixture, second reaction mixture, third mixture, third reaction mixture, fourth mixtures or more mixtures can be agitated and/or incubated from about 1 min to about 15 min, about 5 min to about 45 min, about 1 h to about 12 h, about 5 h to about 15 h, about 10 hours to about 24 hours, about 12 h to about 17 h, about 12 h to about 24 h, about 22 h to about 50 h, or about 24 h to about 72 h.

### EXAMPLES

To provide a better understanding of the foregoing discussion, the following non-limiting examples are offered. Although the examples can be directed to specific embodiments, they are not to be viewed as limiting the invention in any specific respect.

This synthetic methodology describes the synthesis of biocomposites with high-aspect ratio structures. The biocomposites consist of copper and cystine, with either copper nanoparticles (CNPs) or copper sulfate contributing the metallic component. Synthesis is carried out in liquid under biological conditions (37° C.) and the self-assembled composites form after 24 hr. Once formed, these composites are highly stable in both liquid media and in a dried form. The composites scale from the nano- to micro-range in length, and from a few microns to 25 nm in diameter. Field emission scanning electron microscopy with energy dispersive X-ray spectroscopy (EDX) demonstrated that sulfur was present in the NP-derived linear structures, while it was absent from the starting CNP material, thus confirming cystine as the source of sulfur in the final nanocomposites. During synthesis of these linear nano- and micro-composites, a diverse range of lengths of structures is formed in the synthesis vessel. Sonication of the liquid mixture after synthesis was demonstrated to assist in controlling average size of the structures by diminishing the average length with increased time of sonication. Since the formed structures are highly stable, do not agglomerate, and are formed in liquid phase, centrifugation may also be used to assist in concentrating and segregating formed composites. It was discovered that over time, and under biological conditions used for typical cell culturing (37° C. and 5% CO2), stable copper biocomposites could be formed with a high-aspect ratio (linear) structure.

Self-assembly of two types of highly linear biocomposites was discovered to be possible with two starting metal components: copper nanoparticles (CNPs) and copper sulfate, with the common biological component being cystine. Although more complex, so called “urchin” and “nanoflower” type copper-containing structures with nanoscale and microscale features have been previously reported, these were produced under non-biological conditions, such as temperatures of 100° C. or greater.

One of the starting materials utilized for synthesis of nanocomposites, namely CNPs, has been reported previously to be very toxic to cells. It has recently been reported that after the nanocomposites are formed, these structures are less toxic on a per mass basis than the starting NPs. Thus, the synthesis described here may be derived from a biological and biochemical reaction that has utility in stabilizing reactive copper species, both in the sense of transforming the NP form into larger structures and in producing composites less toxic to cells.

In contrast to many other nanomaterial forms which are known to aggregate or clump upon interaction with biological liquid media once formed, the highly linear composites described here avoid aggregation, possibly due to a redistribution of charge which has been previously reported. As detailed in the current work, this avoidance of aggregation is convenient for the purposes of working with the structures once formed.

For this synthesis, use a 37° C. incubator with 5% CO2 and at least 40% humidity. Ensure that such an incubator is available and that it will not be repeatedly disturbed over the period of synthesis (approximately 24 hr). Repeated opening and closing of the incubator will certainly cause temperature fluctuations which may result in altered synthesis of the nanocomposite structures.

It was discovered that cystine, a supplemented component of the original cell cultures, when combined with CNPs under the correct biological conditions, could result in transformation of the nanoparticulate form into a highly linear biocomposite that contained both the metal and biochemical components as indicated by EDX analysis by scanning electron microscopy of the prepared samples. Thus, sulfur, which is not present in the starting CNP material, shows a prominent peak in the synthesized biocomposites, indicating that both copper and components of the biochemical material cystine are essential for the formed linear structures.

It was further shown that by using similar synthesis conditions, but replacing CNPs with copper sulfate, highly linear biocomposites could also be formed. In fact, synthesis using copper sulfate and cystine tended to result in “cleaner” end products, in that no unreacted CNPs were ever present, since copper sulfate is fully soluble in water, which was used as a solvent in all of the syntheses. Further, copper-sulfate biocomposites distinguished themselves from CNP-derived composites in retaining a blue color which was apparent upon centrifugation of the material.

The steps can include: 1) good dispersion by sonication of starting materials in the case of synthesis incorporating CNPs; 2) use of freshly prepared CNPs, copper sulfate, and cystine for effective synthesis of composites; 3) allowing synthesis in the flask to remain undisturbed in the incubator for at least 6 hours; and 4) avoiding “over-reaction” conditions in which branched “urchin”-type, aggregated composites form.

After synthesis is completed, it was shown that sonication of the structures can be utilized effectively to decrease the average size (length) of the structures. Sonicating to make smaller structures may aid in applications such as cell uptake or other biocomposite-cellular interactions. As has been previously reported, charge stabilization of the CNPs during this synthesis by combining with cystine altered the measured zeta potential from positive to a less charged (negative) form. This change in charge may help explain why the formed composites show very little aggregation in the dried form or liquid media, which makes handling of the structures much more convenient.

Since the synthesis of these CNP-derived and copper sulfate-derived structures is carried out in liquid media, it is anticipated that the process may be highly scalable, meaning that with the correct ratio of components and synthesis conditions, the milliliter synthesis recipe used here could be scaled up or down to include many hundreds of milliliters or more, and would thus be expected to yield more final product, as well. Due to the metal (copper) component of these biocomposites, it is quite straightforward to concentrate synthesized product by centrifugation. Biocomposites formed from CNP starting material retain a darker color once concentrated into a pellet, possibly due to unreacted copper oxide nanoparticles. In comparison, copper sulfate biocomposites when concentrated into a pellet have a blue color, consistent with properties of copper sulfate with various levels of hydration.

In an embodiment, the method of making the one or more copper composition can be scalable in the sense that the self-assembled linear structures scale from the nano-scale to the micro-scale as shown using electron microscopy and traditional white light microscopy. In another embodiment, the method of making the one or more copper composition and/or copper structures can include spacers or tagging agents into the linear composites reported here to provide production of larger structures and/or enhancements for imaging.

The green synthesis of copper structures endeavors to reduce the use of high energy methods with those that may include lower temperatures and pressures, use of natural products, and bottom-up self-assembly. Here is described the generation of metal-organic biohybrids (MOBs) with nanoscale features synthesized at physiological (37° C.) and room temperature (25° C.). These MOBs utilized the naturally occurring amino acid dimer cystine as the biological component, and a series of metals, including copper, silver, and cobalt. The copper- and silver-based nanomaterials generated were distinct in size and shape. Copper formed elongated high-aspect ratio structures. In contrast, the self-assembly of cystine and silver formed nanoparticles which are designated as AgCysNPs, and cobalt formed particles which are designated as CoMOBs. Both cobalt and silver could be combined with copper in the same reaction vessel to carry out green synthesis of different nanomaterials simultaneously. Post-synthesis the polarization of light by CuHARS provided one measure to distinguish the size and shape of different MOBs generated simultaneously.

Unique to this synthesis compared to many other nanomaterials is that copper and silver MOBs can be synthesized at physiological temperature (37° C.). This is a benefit for green nanomaterials synthesis, since the energy footprint for self-assembly processes at 37° C. which was used, is much less than for many other reported green procedures which use elevated temperatures, often for extended periods of time. Other work for example has reported production of nanomaterials at room temperature, but use harsh chemicals or top-down energy driven processes such as multiple grinding steps and sonication. Other green synthesis methods for nanomaterials include the use of biological mediators such as bacteria and fungi, or use natural materials such as plant extracts. These biological methods have benefits, but may also involve complex steps that could provide challenges for scaling up production. Further, due to variability in the growth and harvesting of the biological component (for example plant extracts), different synthesis batches might be expected to vary. In contrast, the single biological component used in the green synthesis method is the natural amino acid dimer cystine. We isolated the pure, single component as essential to the green synthesis method, in a step-by-step elimination of all other factors isolated from cell culture conditions where the original discovery was made. Thus, copper structures were made by a green synthesis and defined stoichiometry of metal or metal salts with the single biological component, cystine. This is done without the need of enzymes or microbes or other co-factors, which may be present when harvesting green components used from plant extracts or active bacteria or fungal preparations.

The copper structures are biohybrids due to their stable incorporation of a biological component (cystine), and a non-biological metal such as copper (CuHARS) or silver (AgCysNPs). Key to the discovery of MOBs was the demonstration that CuHARS self-assembled from either copper nanoparticles or copper sulfate, yet both forms contained sulfur upon elemental analysis using EDX by scanning electron microscopy. This finding indicated that the stable self-assembled CuHARS had incorporated sulfur from the cystine used during the synthesis, and/or from the monomer form of cystine, cysteine, which also contains sulfur.

Due to their differences in size and shape, the different MOBs using copper or silver (CuHARS and AgCysNPs, respectively), also offer different avenues for continued green synthesis directions post-synthesis. AgCysNPs remain as nano-scale colloids for long periods of time, which provides potential benefits for optics and coatings. However, it makes concentration of the material more difficult. In contrast, CuHARS settle rather quickly, without agglomeration, providing further green synthesis steps in the application of this material. Centrifugation may also be used to accelerate the process. Due to the very limited aggregation of CuHARS, the material may be separated by size by settling over time (or by centrifugation), and then easily dispersed almost instantaneously by vortexing or by simple inversion (mixing) of the material in water.

Since the MOBs described are generated via a bottom-up, self-assembly process, consistency of temperature is a consideration, but to further explore the diversity and flexibility of the method, green synthesis of MOBs was compared at 37° C. vs 25° C. This consideration for green synthesis of nanomaterials at modest temperatures would be a beneficial driver when considering scaling up production of a material, since the range of manufacturing temperatures available between 37° C. and 25° C. could encompass large arrays of production vessels. Insight into these green synthesis methods could limit (or minimize) the need for synthesis ovens, and thus diminish the electricity footprint.

One aspect of green synthesis methods in the future might consider simultaneous generation of materials in the same vessel. The synthesis of copper MOBs and silver MOBs was previously shown in separate vessels. Here it is demonstrated that silver or cobalt may be combined with copper in the same vessel to create new MOBs. Once generated, the distinct size and shapes of copper, silver, and cobalt MOBs can aid in their isolation and identification: for example, CuHARs readily polarize light as shown under microscopy. Previously, different nanostructures combining copper and other metals have been described using the co-precipitation technique, but this often involved high-temperature heating steps to destroy the organic component of materials used during synthesis or sequential doping steps without organic materials. In the methods described here to generate MOBs, all steps were carried out at 37° C. or below, and in this manner, organic materials such as cystine used in the synthesis were retained. Maintenance of physiologically permissive temperatures during green synthesis of nanomaterials may have advantages not only for biologically derived manufacturing components such as were shown here for cystine, but also production of nanomaterials using intact biological entities and systems such as plants, fungus, bacteria, and yeast. Although it was not optimized for such, the initial discovery of the self-assembly of CuHARS was made in an intact, physiologically maintained cell culture system, and was then simplified to isolate the essential biological component for the biohybrid, in this case the amino acid dimer cystine.

It has been confirmed that the synthesized materials contain organic ligand components, most likely, the starting amino acid dimer cystine and/or the monomer amino acid form, cysteine. This was verified for multiple forms of the MOBs. For copper, it was shown that CuHARS could be formed using copper nanoparticles or copper sulfate. Upon elemental analysis, CuHARS generated from both copper starting materials included sulfur, which was not found in the copper nanoparticle starting material used. Therefore, sulfur in the stable CuHARS formed is most likely in the form of sulfur-containing cystine or cysteine, used during the synthesis. Further support of organic ligand components in CuHARS comes from the presence and increase in carbon and nitrogen peaks in the stable biohybrid compared to the starting materials. Elemental analysis of AgCysNPs and CoMOBs is also consistent with the presence of organic ligand components, and due to the particulate nature of both AgCysNPs and CoMOBs, the organic ligand components indicated by the presence of sulfur and carbon is consistent with a capping/stabilization action by the cystine or cysteine used in the synthesis. Cysteine has been used to synthesize stabilized silver nanoparticles and cobalt-iron nanoparticles, but the use of a dimer cystine as a stabilizing agent has not before this.

The oxidation states of copper and cobalt used here were in the +2 state at the beginning of synthesis, and all metal salts were in the sulfate form. Copper, having possible oxidation states of +2 and +1, is known to bind to cystine and cysteine, and under the reducing conditions of sodium hydroxide used here, cystine or cysteine could bind to copper via deprotonated carboxylate groups. This could lead to a Cu(Cys)2 form, as suggested for cysteine. Here, since a cystine was utilized, a Cu(Cys)2 with Cys being the dimer cystine, could provide a mechanism whereby CuHARS are formed and generate the highly elongated (high-aspect ratio) structures shown. However, since cystine also contains sulfur, if thiol groups are deprotonated as is suggested for cysteine, copper binding to sulfur may be preferred over carboxylate. Thus, it suggests here a mechanism for CuHARS formation whereby copper competes with both sulfur and carboxylate groups of cystine, and this provides length (elongation), and width (3-dimensionality) for the growing CuHARS.

The formation of MOBs using the metals silver and cobalt combined with cystine result in particulate forms, rather than the elongated CuHARS of copper. This may be due to the +1 oxidation state for silver, whereby cystine provides a stabilizing and capping function, surrounding a core of silver to form nanoparticles. The bio-reducing capabilities of many compounds extracted from biologicals has been previously reported for generating silver nanoparticles. Here, it was successful in generating silver nanoparticles using the amino acid dimer cystine.

In contrast to copper and silver, cobalt has oxidation states of +2 or +3, and has been shown to bind strongly to the sulfur atoms of cysteines in observed and computational studies; oxidation phenomena leading to Co(III) in free or complexed form were never observed in these studies. Thus, cobalt under these conditions resulted in large particulates, likely capped by the cystine, or the breakdown product of cysteine. It therefore remains likely that for both silver and cobalt, under the conditions used here for MOBs self-assembly, cystine serves as a stabilizing/capping agent, whereas for copper it serves as a linker, providing an elongation factor that results in high-aspect ratio copper MOBs.

It was demonstrated that the MOBs could be co-synthesized in the same vessel using two combinations: copper and silver, and copper and cobalt. Due to the light polarization properties of CuHARS, co-synthesis of MOBs which include copper may be separated on the basis of size and shape of the materials obtained, and verified using light polarization and digital microscopy.

This facile green synthesis of multiple types of nanomaterials in the same vessel may further advance the benefits of self-assembly using minimal energy. The disclosed green synthesis methods generated microscale and nanoscale materials at both 37° C. and at room temperature, providing a smaller energy footprint for nanomaterials production. In one case, where cobalt and copper were combined in the same vessel, temperature differences contributed to selective green synthesis of cobalt particles alone (at 37° C.), vs. a mixture of cobalt particles and CuHARS, which occurred at room temperature. Thus, under the conditions carried out here, modest temperatures in the range of 37° C.-room temperature, may be used to selectively produce different types of nanomaterials using green synthesis.

The following protocol is used as an example for reaction in a 25 cm² cell culture flask using 7 μL of cystine, 6,643 μL of sterile water, and 350 μL of CNPs. Prepare a 2 mg/ml solution of copper nanoparticles by weighing out at least 2 mg of CNPs. Wear disposable gloves during this step to prevent possible contact of CNPs with skin. Place the nanoparticles in an empty sterile 16 ml glass vial.

Prepare all materials fresh before the start of an experiment, by adding solid materials to solvents right before synthesis is to begin. Keeping stock solutions of cystine and copper starting materials in liquid for long times before the experiment is not recommended and may lead to variable results.

To the vial containing CNPs, add sterile deionized water in the appropriate volume to make a 2 mg/ml solution and vortex the solution for 20 sec to provide dispersion of the nanoparticles before synthesis starts (at least 1 ml total volume is recommended). Do not fill the vial more than halfway with water as this will inhibit mixing by vortexing. CNPs will quickly settle to the bottom of the vial and will appear dark in color (grey to black).

Sonicate the CNP solution for 17 min at RT to provide maximal dispersion of CNPs before start of synthesis. Periodically check to make sure that CNPs are mixing due to sonication. After a successful sonication, CNPs remain suspended in solution for at least 30 min and the solution will be dark in color.

Weigh out sufficient mass of cystine to make a 72.9 mg/ml solution for the synthesis. Since cystine is not directly soluble in water, place the weighed cystine in an antistatic weighing vessel. To the weighing vessel containing cystine, add sufficient volume of sterile, 1 M NaOH, so that the cystine completely dissolves. For example, dissolve 7.29 mg of cystine completely in 100 μL of 1 M NaOH, to make a 72.9 mg/ml solution.

Working in a sterile tissue culture hood, add 7 μL of cystine with 6,643 μL of sterile water to the sterile synthesis flask first, and let incubate for 30 min in the incubator at 37° C. with the flask cap vented (loose) to provide effective mixing. Resuspend the 2 mg/ml CNP solution by vortexing for 30 sec, since CNPs will have settled after the sonication step.

Add sufficient CNP solution to the synthesis flask (using sterile technique) to maintain the following component ratios: combine 1 parts cystine, 50 parts CNPs, and 949 parts sterile water in a 25 cm² cell culture flask to start the synthesis. For example, for a 7 ml synthesis volume, combine 7 μL of cystine stock solution, 350 μL of CNPs, and 6,643 μL of sterile water. Replace the cap on the flask and tighten so that it is secure.

After combining all components for the synthesis, gently mix in the flask by swirling 4-5 times. Place flask in the CO2 incubator and vent the flask by loosening the cap so that there will be gas exchange in and out of the flask during synthesis. Allow synthesis to run in the incubator for approximately 24 hr. During synthesis, one can observe, with microscopy and by eye, formation of highly linear composites.

The process of formation of the copper structures may happen suddenly in the sense that structures are initially hard to detect, then appearance proceeds quickly to an increasing density. Formation may therefore occur before 24 hr. The process can also be observed by eye once structures become larger and their density increases. While generation of the structures can be observed over time under the microscope and by eye at later time points, continuously interrupting the synthesis conditions and temperature will lead to poor synthesis results.

Terminate synthesis of biocomposites by tightly capping the synthesis flask and storing the vessel in a refrigerator (4° C.). Structures, once generated, remain stable in this form for at least a year. Label the flask with synthesis conditions, including components utilized, date of the synthesis, and incubation time of the synthesis before termination.

Self-assembly synthesis by replacing CNPs with copper sulfate salt was performed. Using sterile technique, dissolve at least 2 mg of copper sulfate in sufficient volume of sterile deionized water to make a 2 mg/ml solution. The copper sulfate crystals easily go into solution at this concentration, but vortex the vial if needed, and inspect by eye to ensure all crystals are dissolved.

After preparation of the copper sulfate, carry out synthesis as described previously, but replacing CNPs with the copper sulfate. Self-assembled nanocomposites using copper sulfate as a starting material were found to be much more consistent in final shape than for structures synthesized from CNPs. Terminate the synthesis of copper sulfate biocomposites as for CNP composites and store them long-term at 4° C. The biocomposites derived from CNPs and from copper sulfate were characterized by white light microscopy and electron microscopy.

For characterization and inspection of biocomposites post-synthesis by white light microscopy, use an inverted microscope as composites will settle to the bottom surface of the flask within a few minutes of laying the flask flat, and can then be brought into focus. Use the bright field setting on the microscope to maximize contrast between biocomposites and the liquid medium. Composites derived from CNPs and copper sulfate will both appear clear to opaque in color, but unreacted CNP aggregates will appear very dark in color. Use a digital camera connected to the microscope to capture images of the composites. A range of lengths for the individual structures will be observed.

For characterization and inspection of biocomposites post-synthesis and after storage at 4° C., allow flasks to come to room temperature for at least 15 min as flasks will form condensation initially upon removal from refrigerator, which will obscure effective focusing while carrying out microscopy imaging. After allowing equilibration to RT, wipe the top and bottom surfaces of the flask with a clean paper towel to maximize microscopy imaging quality.

When working with or imaging composites that have been stored long-term, vortex the flask for 30 sec to dissociate clumps of composites that form while in the refrigerator. After vortexing, inspect the structures with an inverted microscope to ensure that aggregates have dissociated, and repeat vortexing as necessary.

Use inverted white light microscopy to assess the efficacy of the synthesis for a given experiment using CNPs. For example, document the presence or absence of unreacted CNPs in synthesis flasks used for CNP-derived biocomposites from flasks with different parameters such as time of synthesis.

Individual CNPs are too small to observe with a light microscope, but unreacted CNP aggregates will appear as round-shape and dark objects, in contrast to the successfully synthesized CNP-composites which will have a high-aspect ratio, linear form, and will have a range of different lengths. Avoid carrying out synthesis for too long of a period of time before termination, as this will result in highly branched “urchin” type structures, which are difficult to disperse into individual structures once formed.

Use inverted white light microscopy to assess the efficacy of the synthesis for a given experiment using copper sulfate. Since copper sulfate goes fully into solution using this protocol, the solution will appear less dark than the solution from synthesis using CNPs. Document the size and extent of copper sulfate composites by comparing flasks with different synthesis conditions such as time of synthesis before termination.

Successfully synthesized composites will show a range of different lengths. Avoid carrying out synthesis for too long of a period of time before termination, as this will result in highly branched aggregates of composites, some of which will be “urchin-like” in structure, and which are difficult to disperse into individual structures once formed.

To concentrate biocomposites post-synthesis, centrifuge solutions of composites in a centrifuge tube. Add 6 ml of either CNP-derived structures or copper sulfate-derived structures to a 15 ml centrifuge tube. Centrifuge for 10 min at 500×g at RT to form a pellet. For smaller volumes, add 500 μl of structures in solution to 0.6 ml sized tubes. Centrifuge at 2,000×g at RT for at least 10 min to form a pellet.

After centrifuging for sufficient time (at least 10 min for microfuges), save the observable pellet at the bottom of the tube where the structures are concentrated by carefully removing the supernatant liquid above the pellet. Biocomposite structures derived from copper sulfate appear blue in color and structures derived from CNPs are darker (grey to black).

Add more composites to this tube and repeat the process in the same tube to concentrate structures if desired. To disperse the concentrated pellets, add the desired volume of solution to the tube, and vortex for 10-30 sec.

Sonicate structures once formed, to move the average population size (lengths) of the structures to lower values. Place structures in sterile deionized water and sonicate for at least 10 min. Using this process, over time, structures become fragmented and smaller in average length. Document changes in composite sizes with different sonication times using an inverted white light microscope and digital camera.

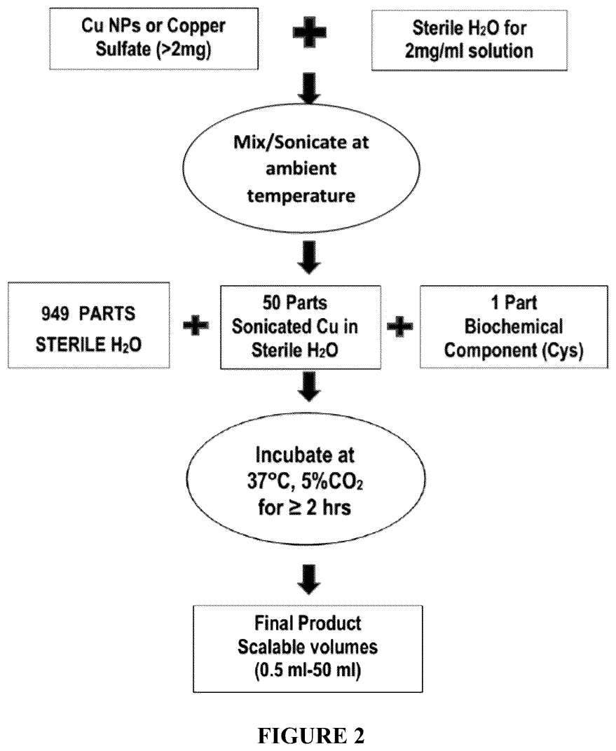

FIG. 2 shows a flow-chart for an embodiment to make the one or more biocomposites. CNPs or copper sulfate as starting materials are combined with sterile water to form a 2 mg/ml solution, this solution is mixed and sonicated to provide an even mixture, and this copper solution is then mixed in the following ratio for synthesis:949 parts sterile water:50 parts copper mixture:1 part cystine stock solution. The actual volumes may be increased or decreased according to these ratios to scale up or scale down the final synthesis yield. After incubation for at least 2 hr as indicated, linear biocomposite structures are formed which over time can be observed under normal white light microscopy or by eye as the liquid solution changes in appearance.

CuHARS were generated as described above using copper sulfate. Copper (II) sulfate pentahydrate, cobalt (II) sulfate, and sodium hydroxide were from Sigma-Aldrich (St. Louis, MO). Cysteine hydrochloride monohydrate (catalogue #C6852) and L-cystine (catalogue #C7602) were both from non-animal sources and were used as prepared from Sigma-Aldrich. Silver sulfate was from Alfa Acsar (Haverhill, MA). The synthesis was terminated by moving synthesis vessels to 4° C. for long-term storage.

A Beckman-Coulter DU800 spectrophotometer was used to scan the absorbance of samples from 200-800 nm (or as indicated) in a volume of 700 μL. Scanning in the ultraviolet region utilized quartz cuvettes. Both amino acids were solubilized at 7.29 mg per 100 μL of NaOH, and then 14 μL of the concentrated solution added to 7 ml of deionized, sterile water as per the conditions used for MOBs synthesis.

Generation of AgCysNPs. AgCysNPs were generated in a total volume of 7 ml in general following the procedure described to generate CuHARS and including cystine, but replacing copper with a 2 mg/ml solution of silver sulfate. In some cases, a final concentration of 1 mM HCl was included in the synthesis vessel as previously described. Silver solution was used in 2 parts (700 μL), or 3 parts (1,050 μL) of the total volume as indicated, and paired with 2 parts (14 μL), or 3 parts (21 μL) of cystine solution, respectively.

It can be possible to generate multiple MOBs simultaneously. In some cases as indicated, CuHARS and AgCysNPs, or CuHARS and cobalt MOBs (COMOBs), were generated simultaneously. Generation of multiple MOBs types simultaneously was carried out in a total volume of 7 ml in general following the procedure described to generate CuHARS alone and including cystine, but also including with copper, a 2 mg/ml solution of silver sulfate or cobalt sulfate, respectively.

Separation of AgCysNPs with centrifugation. An Eppendorf model 5804R centrifuge was used at 2,500 ref for ten minutes to separate larger AgCysNPs (pellet) from smaller nanoparticles (supernatant), using 15 ml polystyrene tubes (Perfector Scientific, Atascadero, CA), as indicated.

For spectrophotometry scanning of AgCysNPs, NPs were subjected to 3 cycles of centrifugation using 4 ml of deionized water with retention of the pellet, as indicated. Final pellet was then resuspended in 3.5 ml of deionized water and analyzed by spectrophotometry from 200-800 nm.

Coffee-ring formation. Four microliters of separated AgCysNPs or other materials as indicated were spotted onto a glass microscope slide, and dried at 50° C. Dried spots were then imaged for coffee-ring formation using an inverted Leica DMI6000 B microscope with Leica DFC500 color camera or an inverted Olympus IX51 microscope with Olympus DP71 camera as indicated.

Dynamic Light Scattering (DLS) measurements were taken on a Malvern Zetasizer Nano ZS instrument and associated analysis carried out using Zetasizer software (Ver. 7.1). Samples were sonicated for 5 minutes using a Branson model 1800 sonicator before taking DLS measurements.

Scanning Electron Microscopy (SEM, Hitachi, Japan) was carried out on samples using a Hitachi FESEM by drying samples onto silicon wafers and then carrying out microscopy at the indicated magnification. Energy dispersive X-ray spectroscopy (EDX) analysis was used to identify elemental components of the scanned nanomaterials.

Magnetic Susceptibility. A magnetic field was applied using one or two neodymium ring magnets (Applied Magnets, Plano, TX), as indicated. The measured average local magnetic field was 107±16 mTesla on the large opening side of the magnet, and 150±49 mTesla for the small opening side of the magnet as measured by an F.W. BELL Gauss/TeslaMeter (Model 5080).

The comparative generation of AgCysNPs at 37° C. and 25° C. were assessed, over a period of 5 hours. Substantial yield was achieved, with slight color variations observed for the colloidal nanoparticle suspensions. Larger and smaller components of the self-assembled AgCysNPs could be separated in the pellet and supernatant, respectively, with a relative centrifugal force (rcf) of 2,500 for 10 minutes.

Dynamic light scattering (DLS) analysis demonstrated distinct populations of generated AgCysNPs under the different conditions and in the centrifuged pellets vs. isolated supernatants.

As expected, supernatants contained much smaller nanoparticles than found in the pellets. Inclusion of HCl also resulted in silver nanoparticles with average diameter of 30 nm.

Here, it was shown that both 37° C. and room temperature conditions could be successfully used to carry out green synthesis of silver nanoparticles. Scanning electron microscopy (SEM) was used to verify the nanoscale features of the synthesized silver nanoparticles, and EDAX analysis demonstrated the elemental presence of sulfur and silver, indicating the biohybrid nature of the material.

Generation of AgCysNPs under the different conditions may also be assessed optically using the formation of the coffee-ring effect, as described previously to compare AgCysNPs vs. CuHARS. Here it was found that AgCysNPs generated under all conditions demonstrated a coffee-ring effect upon drying. Lower magnification of dried samples showed wider views of the coffee-ring effect.

Spectrophotometry revealed a slight shift in absorbance values for the different silver: cystine ratios, and for 37° C. and 25° C. synthesis conditions. It was found that when using the dimer of the amino acid, cystine for MOBs synthesis, that absorbance at 390 nm and in general, was greater for room temperature (25° C.) synthesis conditions than for 37° C. synthesis. No specific peak was observed, but rather a broad, generalized increase in absorbance with decreasing wavelength, consistent with what was reported previously, demonstrating a strong increase in the range of 400-200 nm where cystine absorbs.

When comparing the concentration of the dimer cystine used here to the amino acid cysteine in the thiolated form (dissolved in NaOH), it was found differences in absorbance spectra and consistent with prior results comparing these two sulfur-containing amino acids. However, when scanning the prepared AgCysNPs after 3 cycles of washing with water and centrifugation, no distinct peaks were detected (800-200 nm), although some shoulder regions in the scans did emerge.

Considering the light sensitivity of silver salts and silver nanoparticles, it is hypothesized that darker AgCysNPs product generated at room temperature. Room temperature synthesis was not carried out in a darkened oven, while 37° C. synthesis was carried out in a darkened oven. Identical synthesis conditions were carried out using a darkened 37° C. oven and one maintained at room temperature (25° C.). Under these conditions, it was found that identical ratios of silver to cystine resulted in similar colored solutions at 37° C. and 25° C., with notable loss of the darker product at room temperature. In fact, compared to conditions where room temperature synthesis occurred in the presence of light, room temperature synthesis in a darkened oven resulted in lighter colored solutions than the 37° C. condition. This was the first demonstration of self-assembly synthesis using copper and silver in the same vessel, carried out at 37° C. and 25° C. as indicated.

A difference in color is noted in the products produced simultaneously, with silver and copper co-synthesis at room temperature resulting in a darker (reddish) color for both pellet and supernatant, and synthesis at 37° C. resulting in a brown pellet with more yellow colloidal supernatant. Coffee ring drying of the copper and silver co-synthesis revealed a mixture of silver particulates and high-aspect ratio copper materials. In addition to the separation of materials by shape and drying pattern, here it was observed in the copper/silver mixture, a clear polarization pattern which was previously described only in pure CuHARS samples. Due to size and shape, the polarizing materials are consistent with CuHARS, whereas the AgCysNPs are non-polarizing.

In addition, the reactions were carried out for combined cobalt and copper MOBs synthesis using cobalt sulfate. Synthesis was carried out as indicated at 37° C. and 25° C., and resulted in distinct, whitish-colored products, with 37° C. synthesis conditions resulting in a darker product compared to room temperature.

As was done for the combination of silver and copper samples, the combined cobalt and copper samples were applied to microscope slides for coffee-ring analysis and the cobalt MOBs were shown to be particulate and non-polarizing, whereas CuHARS were elongated and polarizing.

It was interesting to note that under conditions of 37° C. vs room temperature, cobalt nanoparticles were favored. In contrast, under the same component ratios, CuHARS only appeared when green synthesis was carried out at room temperature. Increasing all synthesis components resulted in the greatest amount of material for both cobalt and copper when carried out at 37° C. The CoMOBs particles formed ranged from nanoscale to microscale. Elemental analysis of the generated CoMOBs indicated a biohybrid with both cobalt and sulfur appearing as prominent components of the material. MOBs generated using CuHARS, scale from the nano- to the micro-with nanoscale diameters and microscale length and were also shown in the same sample synthesizing copper and cobalt MOBs simultaneously. EDX analysis of CuHARS demonstrated sulfur and copper self-assembling as a biohybrid, with sulfur coming from the amino acid dimer cystine, combining with the non-biological metal component of copper, and as shown here when synthesized simultaneously with cobalt.

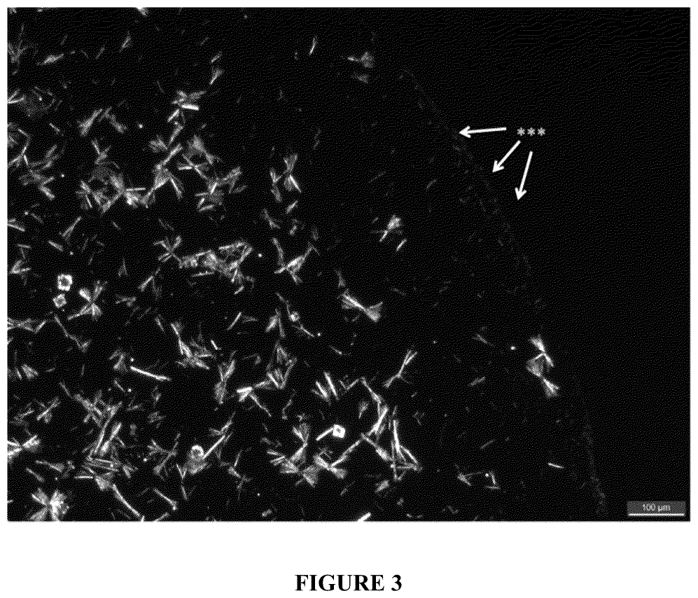

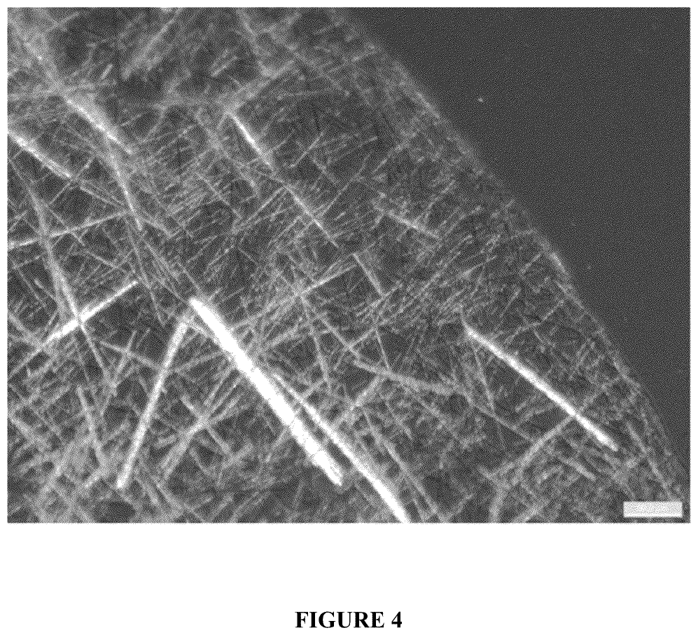

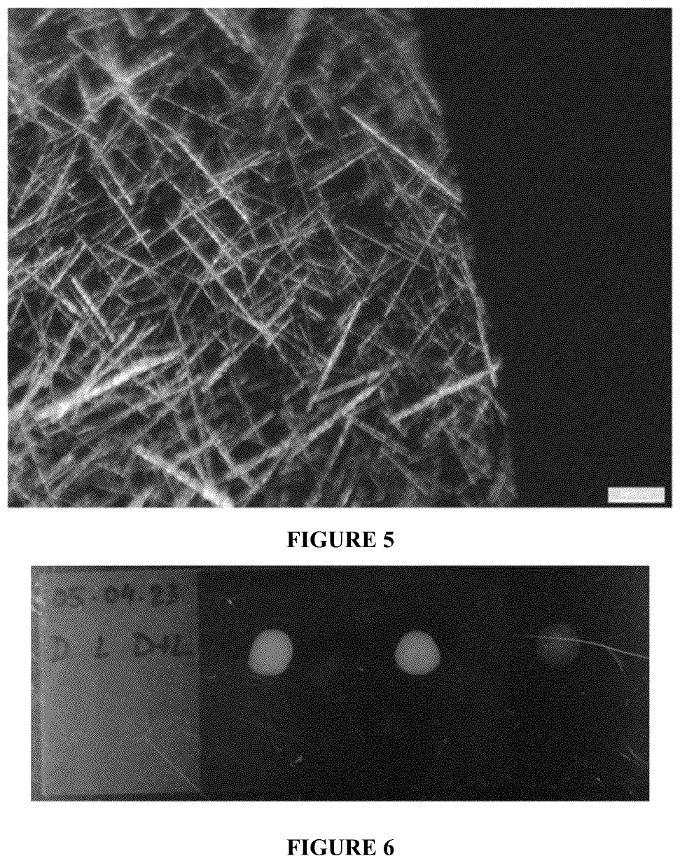

High aspect ratio composites with biological and metal components were synthesized. Scanning electron microscopy (SEM) and Transmission Electron Microscopy (TEM) revealed linear morphology and smooth surface texture. SEM, TEM, and light microscopy showed that composites have scalable dimensions from nano- to micro-, with diameters as low as 60 nm, lengths exceeding 150 μm, and average aspect ratio of 100. The structures are stable, remaining intact for over one year in dried form and in liquid, and did not aggregate, in contrast to metal nanoparticles such as iron and copper. FIGS. 3-5 show magnified images of the edge of a droplet of the copper compositions containing D+L cystine, D-cystine, and L-cystine, respectively. FIG. 6 shows an image of spotted and dried droplets on a microscope slide of three formulations of CuHARS using D-cystine, L-cystine, and racemic (D+L)-cystine. In FIG. 6, the CuHARS droplets appear as bright round objects, or faint object in the case of (D+L). For this image, the microscope slide was placed on a linear polarized transparent sheet which was laying on a lightbox. Then, a second linear polarized transparent sheet was held above the microscope slide, and light passing through was imaged using a smartphone camera. Due to the polarization alignment of the two polarized sheets, CuHARS appear white against a dark background, enhancing signal-to-noise ratio, thus greatly improving the ability to quantify polarizing material. It should be noted that the white scratches are due to scratches on the protective coverings on the polarized films.

Many metal nanoparticles are toxic to cells, limiting their use for biological applications. The bio-metallic composites characterized here showed lower toxicity compared to their precursor metal nanoparticles in brain tumor cell cultures. Due to these more biocompatible properties, the ability of the composites to interact with cells was tested. Zeta potential analysis indicated that composites carry a net negative charge (−24.3±2.2 mV), while the starting metal nanoparticles measured (43.3±2.4 mV). The composites were labeled with poly-1-lysine fluorescein isothiocyanate (PLL-FITC), which shifted the potential to 3.5±2.9 mV.

Also, it was observed that very small volumes of the copper composition (2-4 μL) has been shown to respond to specific chemical reactants, such as acids and bases, without being altered by control solutions (water). This provided the basis for a potential degradable, fast-response sensor for liquid chemical and potentially volatile chemical sensing that would provide high signal: noise output.

One of ordinary skill in the art will readily appreciate that alternative but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. It should also be appreciated that the numerical limits may be the values from the examples. Certain lower limits, upper limits and ranges appear in at least one claims below. All numerical values are “about” or “approximately” the indicated value, and consider experimental error and variations that would be expected by a person having ordinary skill in the art.

What is claimed is:

1. A chemical sensor comprising:
a substrate;
a film, wherein the film comprises one or more copper cystine biocomposites, and wherein the film is at least partially disposed on one or more surfaces of the substrate;
a light source, wherein the light source irradiates the one or more copper structures;
and a detector, wherein the detector receives light reflected from the one or more copper cystine biocomposites, and wherein the detector detects a change in light polarization when the one or more copper structures contact one or more chemical species.

2. The chemical sensor of claim 1, wherein the one or more copper cystine biocomposites comprise D-cystine.

3. The chemical sensor of claim 1, wherein the one or more copper cystine biocomposites comprise L-cystine.

4. The chemical sensor of claim 1, wherein the one or more copper cystine biocomposites have with an aspect ratio of about 1 to about 100.

5. The chemical sensor of claim 1, wherein the one or more copper cystine biocomposites have an average diameter about 60 nm to about 60 microns.

6. The chemical sensor of claim 1, wherein the one or more copper cystine biocomposites have a content of cystine from about 20 wt % to about 80 wt %.

7. The chemical sensor of claim 1, wherein the one or more chemical species are selected from a list comprising: microplastics, acids, bases, hydrogen peroxide, water vapor, and nitric oxide.

8. The chemical sensor of claim 1, wherein the light source emits white light.

9. A method for sensing the presence of a chemical species, wherein a method comprises:
making one or more copper structures, wherein the method of making the one or more copper structures comprises:
contacting one or more copper species, one or more cystines, one or more carrier fluids to make a first mixture;
agitating the first mixture to make an agitated first mixture; and
incubating the agitated first mixture to make the one or more copper structures;
applying a film of the one or more copper structures to one or more surfaces of a substrate;
irradiating the one or more copper structures with a light source;
detecting a change in light polarization when the one or more copper structures contact the chemical species.

10. The method for sensing the presence of a chemical species of claim 9, wherein the one or more cystines comprise D-cystine.

11. The method for sensing the presence of a chemical species of claim 9, wherein the one or more cystines comprise L-cystine.

12. The method for sensing the presence of a chemical species of claim 9, wherein the one or more copper species comprise copper nanoparticles.

13. The method for sensing the presence of a chemical species of claim 9, wherein the one or more copper species comprise copper sulfate.

14. The method for sensing the presence of a chemical species of claim 9, wherein the incubating the agitated first mixture was incubated for a time of greater than 2 hours, and wherein incubating the agitated first mixture was incubated at a temperature from about 37° C. to about 40° C.

15. The method for sensing the presence of a chemical species of claim 9, wherein the one or more copper structures comprise copper particles with an aspect ratio of about 1 to about 10.

16. The method for sensing the presence of a chemical species of claim 9, wherein the one or more copper structures have a content of cystine from about 20 wt % to about 80 wt %.

17. The method for sensing the presence of a chemical species of claim 9, wherein the one or more copper structures comprise copper particles with an average diameter about 5 nm to about 500 nm.

18. The method for sensing the presence of a chemical species of claim 9, wherein the one or more chemical species are selected from a list comprising: microplastics, acids, bases, water vapor, nitric oxide, and hydrogen peroxide.

19. A method for a chemical sensor, wherein the method comprises:
making one or more copper cystine biocomposites, wherein a method of making the one or more copper cystine biocomposites comprise:
contacting one or more copper species, one or more cystines, one or more carrier fluids to make a one or more copper cystine biocomposites;
applying a coating of the one or more copper cystine biocomposites to one or more surfaces of a substrate;
contacting the one or more copper cystine biocomposites with a known chemical species;
irradiating the one or more copper structures that is contact with the known chemical species with a light source;
measuring a first polarization of a reflected light from the one or more copper cystine biocomposites that is in contact with a known chemical species;
contacting the one or more copper cystine biocomposites with an unknown chemical species;
irradiating the one or more copper structures that is in contact with the unknown chemical species with a light source;
measuring a second polarization of the reflected light from the one or more copper cystine biocomposites that is in contact with the unknown chemical species;
comparing the first polarization with the second polarization.

20. The method for a chemical sensor of claim 19, wherein the one or more cystines comprise D-cystine.
